(19) United States
(12) Patent Application Publication
(10) Pub. No.: US 2014/0260097 A1
(43) Pub. Date: Sep. 18, 2014

(54) AUTOMATED CRIMPING OF TRANSCATHETER HEART VALVES

(71) Applicant: Edwards Lifesciences Corporation, Irvine, CA (US)

(72) Inventors: Neal Avery, Mission Viejo, CA (US); Hao Shang, Fullerton, CA (US); Julie A. Benton, Mission Viejo, CA (US); Bin Tian, Irvine, CA (US)

(73) Assignee: Edwards Lifesciences Corporation, Irvine, CA (US)

(21) Appl. No.: 14/211,775

(22) Filed: Mar. 14, 2014

Related U.S. Application Data
(60) Provisional application No. 61/800,112, filed on Mar. 15, 2013.

(51) Int. Cl. A61F 2/24; B65B 5/04

(52) U.S. Cl. CPC A61F 2/2427; B65B 5/04
USPC 53/428; 72/452.3; 72/367.1

(57) ABSTRACT

The present disclosure is directed to embodiments of automated crimping systems, methods of crimping prosthetic valves, and methods of using automated crimping systems to crimp prosthetic valves. In some embodiments, an automated crimping system includes a crimper controller, a rotary actuator, an outer crimper housing, and a crimper mechanism mounted within the outer crimper housing. In some embodiments, an automated method of crimping a prosthetic valve includes inserting a prosthetic valve into an automated crimping system, activating the automated crimping system, and removing the crimped prosthetic valve from the automated crimping system. In some embodiments, a method of crimping a prosthetic valve comprises partially crimping the prosthetic valve at a first speed to a partially crimped configuration and further crimping the prosthetic valve at a second speed.

### RELATED APPLICATION

This application claims the benefit of U.S. Patent Application No. 61/800,112, filed Mar. 15, 2013, which application is hereby incorporated herein by reference in its entirety.

### FIELD

The present disclosure relates to implantable expandable prosthetic devices and, more particularly, to systems and methods for crimping a prosthetic device.

### BACKGROUND

The human heart can suffer from various valvular diseases, which can result in significant malfunctioning of the heart and ultimately require replacement of the native valve with an artificial valve. There are a number of known artificial valves and a number of known methods of implanting these artificial valves in humans. Because of the drawbacks associated with conventional open-chest surgery, percutaneous and minimally-invasive surgical approaches are in some cases preferred. In one such technique, a prosthetic valve is configured to be implanted in a much less invasive procedure by way of catheterization. For example, collapsible transcatheter prosthetic heart valves can be crimped to a compressed state and percutaneously introduced in the compressed state on a catheter and expanded to a functional size at the desired position by balloon inflation or by utilization of a self-expanding frame or stent.

A prosthetic valve for use in such a procedure can include a radially collapsible and expandable frame to which leaflets of the prosthetic valve can be coupled. For example, U.S. Pat. Nos. 6,730,118, 7,393,360, 7,510,575, and 7,993,394, which are hereby incorporated herein by reference, describe exemplary collapsible transcatheter prosthetic heart valves.

Some prosthetic heart valves are conventionally packaged in jars filled with preserving solution for shipping and storage prior to implantation into a patient, though techniques are also known for drying and storing bioprosthetic heart valves without immersing them in a preservative solution. The term “dried” or “dry” bioprosthetic heart valves refers simply to the ability to store those bioprosthetic heart valves without the preservative solutions, and the term “dry” should not be considered synonymous with brittle or rigid. Indeed, “dry” bioprosthetic heart valve leaflets may be relatively supple even prior to implant. There are a number of proposed methods for drying bioprosthetic heart valves, and for drying tissue implants in general, and the present application contemplates the use of bioprosthetic heart valves processed by any of these methods. A particularly preferred method of drying bioprosthetic heart valves is disclosed in U.S. Pat. No. 8,007,992 to Tian, et al. An alternative drying method is disclosed in U.S. Pat. No. 6,534,004 to Chen, et al. Again, these and other methods for drying bioprosthetic heart valves may be used prior to using the crimping systems and methods described herein.

One such strategy is to dehydrate the bioprosthetic tissue in a glycerol/ethanol mixture, sterilize with ethylene oxide, and package the final product “dry.” This process eliminates the potential toxicity and calcification effects of glutaraldehyde as a sterilant and storage solution. There have been several methods proposed that use sugar alcohols (e.g., glycerol), alcohols, and combinations thereof in post-glutaraldehyde processing methods so that the resulting tissue is in a “dry” state rather than a wet state, that is, with excess glutaraldehyde. U.S. Pat. No. 6,534,004 (Chen et al.) describes the storage of bioprosthetic tissue in polyhydric alcohols such as glycerol. In processes where the tissue is dehydrated in an ethanol/glycerol solution, the tissue may be sterilized using ethylene oxide (ETO), gamma irradiation, or electron beam irradiation.

More recently, Dove, et al. in U.S. Patent Application Publication No. 2009/0164005 propose solutions for certain detrimental changes within dehydrated tissue that can occur as a result of oxidation. Dove, et al. propose permanent capping of the aldehyde groups in the tissue (e.g., by reductive amination). Dove, et al. also describe the addition of chemicals (e.g., antioxidants) to the dehydration solution (e.g., ethanol/glycerol) to prevent oxidation of the tissue during sterilization (ethylene oxide, gamma irradiation, electron beam irradiation, etc.) and storage. Tissue processed in accordance with the principles disclosed in Dove, et al. are termed, “capped tissue”, and therefore bioprosthetic heart valves which use such tissue are termed, “capped tissue valves”. Capping the glutaraldehyde terminates the cross-linking process by consuming all of the free aldehyde groups, and it is believed that this in conjunction with removing the prosthetic tissue valve from the cross-linking solution (e.g., glutaraldehyde) by storing dry is the most effective way to terminate the cross-linking process.

Once manufactured, packaged, and delivered to a physician, prosthetic valves can be crimped manually by the physician using a disposable crimping device prior to implantation. For example, U.S. Pat. No. 7,530,253, which is hereby incorporated herein by reference, describes an exemplary prosthetic valve crimping device. Manual crimping of prosthetic valves can lead to ergonomic and repeatability issues, however, and one-time use of disposable crimping devices can increase cost and waste. Current crimping systems used for automated and/or repeated crimping of stents cannot be used effectively with many current prosthetic valves having leaflets treated with various chemical compounds, as described above, because the liquid forced out of the leaflets during the crimping process can cause contamination and/or corrosion of components of the crimping system. In some cases, such crimping systems can be difficult or time-consuming to clean. Accordingly, systems for crimping prosthetic valves that are automated, and/or that can be rapidly and simply cleaned, and thereby allow more efficient reuse, are desirable.

Further, the process of crimping a prosthetic valve that includes leaflets treated with various chemical compounds, as described above, can compress the leaflets and thereby force interstitial fluid out of the tissue leaflets. In cases where the interstitial fluid has a relatively high viscosity (e.g., where the leaflets were treated with a chemical agent having a relatively high viscosity, such as glycerol) and the prosthetic valve is crimped relatively quickly, movement of the interstitial fluid through the tissue can generate shear stresses in the tissue, and can thereby damage the leaflets (e.g., by causing delamination), potentially reducing the prosthetic valve's reliability and/or performance. Accordingly, methods of reducing or eliminating the potential damage to biological prosthetic valve leaflets during crimping are desirable.

### SUMMARY

The present disclosure is directed to embodiments of automated crimping systems, methods of crimping prosthetic valves, and methods of using automated crimping systems to crimp prosthetic valves. In some embodiments, an automated crimping system for crimping a prosthetic valve comprises a crimper controller, a rotary actuator in communication with the controller, an outer crimper housing coupled to the actuator and configured to rotate upon actuation of the actuator, and a non-metallic crimper mechanism mounted within the outer crimper housing. The crimper mechanism can comprise an inner housing and crimping teeth in the inner housing, the crimping teeth being operably coupled to the outer crimper housing such that rotation of the outer crimper housing effects movement of the crimping teeth.

In some embodiments, the system further comprises a stationary support configured to support the inner housing in a stationary position. The stationary support can comprise a plurality locking notches that engage mating features of the inner housing. In some embodiments, the system further comprises a torque transmittal assembly coupled to the actuator and to the outer crimper housing, wherein the torque transmittal assembly transmits torque between the actuator and the outer crimper housing. The outer crimper housing can comprise a plurality of grooves having a spiral configuration and each of the crimping teeth can comprise at least one knob constrained to move radially with respect to the crimper mechanism and extending into and interacting with the at least one groove. The crimper mechanism can be mounted within the outer crimper housing such that the crimper mechanism can be removed using only a screwdriver or a hex key (e.g., an ALLEN® wrench (Apex Tool Group, Sparks, Md.)) or another type of hand tool, or by hand without using a tool.

In some embodiments, an automated method of crimping a prosthetic valve comprises inserting a prosthetic valve into an automated crimping system, activating the automated crimping system to crimp the prosthetic valve, and removing the crimped prosthetic valve from the automated crimping system. In some embodiments, a method of crimping a prosthetic valve comprises partially crimping the prosthetic valve at a first speed to a partially crimped configuration, and further crimping the prosthetic valve at a second speed, wherein the second speed is slower than the first speed. The prosthetic valve can be crimped at the second speed to a fully crimped configuration onto a delivery apparatus. In some cases, partially crimping the prosthetic valve takes less than ten seconds and fully crimping the prosthetic valve takes more than two minutes.

In some embodiments, an assembly comprises a sterile crimper mechanism comprising a housing and a plurality of crimping teeth within the housing, and a package containing the crimper mechanism.

The foregoing and other features and advantages will become more apparent from the following detailed description of several embodiments which proceeds with reference to the accompanying figures.

### BRIEF DESCRIPTION OF THE DRAWINGS

FIG. 1 shows an exemplary automated crimping system.

FIG. 2 shows an exploded view of the automated crimping system of FIG. 1.

FIG. 3 shows a rotary actuator of the automated crimping system of FIGS. 1-2.

FIG. 4 shows an exploded view of a torque transmittal assembly of the automated crimping system of FIGS. 1-2.

FIG. 5 shows another view of the torque transmittal assembly of FIG. 4.

FIG. 6 shows an inner shaft assembly of the automated crimping system of FIGS. 1-2.

FIG. 7 shows a stationary locking element of the inner shaft assembly of FIG. 6.

FIG. 8 shows an exploded view of an outer crimper housing of the automated crimping system of FIGS. 1-2.

FIG. 9 shows another view of the outer crimper housing of FIG. 8.

FIG. 10 shows a stationary support of the automated crimping system of FIGS. 1-2.

FIG. 11 shows an exploded view of a disposable crimper mechanism of the automated crimping system of FIGS. 1-2.

FIG. 12 shows another view of the disposable crimper mechanism of FIG. 11.

FIGS. 13-14 show components of the automated crimping system of FIGS. 1-2 in an assembled configuration.

FIG. 15 shows an alternative torque transmittal assembly which can be used with the automated crimping system of FIGS. 1-2.

FIG. 16 shows a sterile package for a crimper mechanism.

FIG. 17 shows two calibrated pin gauges.

FIG. 18 shows a prosthetic valve and a protective sleeve partially installed in the crimping system of FIGS. 1-2.

FIG. 19 shows a prosthetic valve delivery system positioned within the prosthetic valve of FIG. 18.

FIG. 20 shows the delivery system of FIG. 19 extending into the torque transmittal assembly of the crimping system of FIGS. 1-2.

FIG. 21 shows the prosthetic valve of FIG. 18 in a partially crimped configuration.

FIG. 22 shows the prosthetic valve of FIG. 18 in a fully crimped configuration.

FIG. 23 shows the crimped prosthetic valve of FIG. 22 positioned within a protective casing.

FIG. 24 is a flow chart of a method for implanting an expandable prosthetic valve.

FIG. 25 is a flow chart of a method for crimping an expandable prosthetic valve.

FIG. 26 shows a prosthetic valve crimped onto a balloon having a pair of bumpers.

### DETAILED DESCRIPTION

### Automated Crimping Systems

FIGS. 1-14 illustrate one exemplary embodiment of an automated crimping system which can be used to crimp a prosthetic device, for example, a prosthetic heart valve. As shown in FIGS. 1 and 2, an automated crimping system 100 can include a rotary actuator 102, a torque transmittal assembly 104, an outer crimper housing 106, a crimper controller 108, a delivery system support pedestal 110, and a stationary support 112. The various components of the system 100 can be supported on a base plate 170 and a vertical support plate 172, which can be rigidly coupled to one another. A crimper mechanism 134 (FIG. 11) is housed within the outer crimping housing 106. In particular embodiments, the crimper mechanism 134 is removable from the housing 106 for cleaning or replacement. Automated crimping systems shown and described herein can be operated to crimp a prosthetic heart valve automatically, that is, primarily by the force of a machine, rather than a person.

The crimper controller 108 can be used to control the operation of the crimping system 100, as further described below. It can be provided with a computer and a user interface which allows an operator to control the system 100, including by programming various crimping sequences, initiating a crimping sequence, and shutting down the system 100. The user interface can include various known devices for allowing a user to interact with a computer, including a keyboard, mouse, buttons, switches, pedals, speakers, monitors, etc. Various controllers, as well as system support pedestals, appropriate for use in the system 100 are commercially available, for example, from Blockwise Engineering, LLC, of Tempe, Ariz.

FIG. 3 shows the rotary actuator 102, which can be provided in various forms and can be powered by a variety of mechanisms, including a hydraulic, pneumatic, an electric motor, or an internal combustion engine, and which is capable of generating mechanical rotation from any of various sources of power. Pneumatic actuators can be advantageous because they can have no electronic components. Any of various suitable standard electric stepper motors can also be advantageous, because they can be controlled by monitoring a torque exerted by the actuator (which can have a known relationship to the force exerted against the prosthetic valve) or by monitoring displacement of the actuator (which can have a known relationship to the size of an aperture in a crimper mechanism, as described further below). Such measurements can be accomplished, for example, using a strain gauge operatively connected to the actuator 102.

In particular embodiments, the actuator 102 has a rotatable ring 178 having a recess 182 disposed therein, and a hollow axis or bore 180 extending through the ring 178 and the body of the actuator 102. The rotatable ring 178 of the actuator 102 can be coupled to the torque transmittal assembly 104 and the bore 180 can receive an inner shaft assembly 120 (FIG. 6) such that the actuator 102 can induce rotation of the torque transmittal assembly 104 relative to the inner shaft assembly 120, which remains stationary. Various actuators appropriate for use in the system 100 are commercially available. For example, SMC Corporation manufactures various rotary actuators, and as one particular example, the SMC MSQ series of pneumatic rotary actuators can be used in the system 100. The torque transmittal assembly 104 transfers rotational movement of the actuator 102 to the outer housing 106, which in turn effects movement of a plurality of crimping teeth 144 of the crimper mechanism 134.

The rotary actuator 102 can be controlled by the controller 108, and thus can be made to produce a variety of sequences of rotational motion, in accordance with any of various sequences programmed into the controller 108. For example, the controller 108 can be programmed with a series of instructions that call for a random or a predetermined sequence of rotational accelerations and/or movements, which may be performed any number of times or simply repeated until another command is received. The controller 108 controls the orientation of the rotary actuator 102, such as by regulating the flow, release, and/or pressure of compressed air to the actuator 102, thereby causing the actuator 102 to carry out the desired sequence of rotational motions. Based on user input to the controller 108, the controller can control the operation of one or more valves that regulate the amount of compressed air supplied to the actuator.

The torque transmittal assembly 104, best illustrated in FIGS. 4 and 5, can be coupled to the rotary actuator 102 such that the actuator 102 can cause the assembly 104 to rotate along its central longitudinal axis of rotation 136. The torque transmittal assembly 104 can include two outer rings 114a, 114b, one located at each end of the assembly 104 along its longitudinal axis 136, a plurality of rods 116 (four in the illustrated embodiment) extending along the length of the assembly 104 and coupling the rings 114a, 114b to one another, and an outer tube 118, also extending along the length of the assembly 104. One of the rings 114a can be configured to be coupled to the actuator 102. As best shown in FIGS. 4 and 14, for example, the ring 114a can be provided with a pin 184 extending laterally from an opening 185 (FIG. 4). The pin 184 can extend into recess 182 (FIG. 3) of the actuator 102, thereby rotationally coupling the assembly 104 to the actuator 102. As shown in FIG. 14, the other ring 114b can be configured to be coupled to the outer crimper housing 106. The rods 116 can be configured to transmit torque between the rings 114a and 114b and thereby between the actuator 102 and the housing 106. The rings 114a, 114b and rods 116 can be made of any of various suitable materials, including metallic materials, for example, stainless steel. The outer tube 118 can substantially enclose the space between the two rings 114a, 114b and can be made of a transparent material, for example, a polycarbonate material.

Referring to FIGS. 6, 7, and 14, the inner shaft assembly 120 can be coupled to the actuator 102 such that it remains stationary as the actuator 102 induces rotation of the torque transmittal assembly 104. The inner shaft assembly 120, in cooperation with the stationary support 112, holds the housing of the crimper mechanism 134 stationary, as further described below. The inner shaft assembly 120 can include a stationary shaft 122 and an inner tube 124 coupled to one end of the stationary shaft 122. As best shown in FIG. 14, the shaft 122 has a first end portion 123 that extends through the bore 180 in the actuator 102 so as to be supported by the actuator. The shaft also extends through outer ring 114a and has a second end portion 125 that extends into and is secured to a first inner coupling ring 126a. The inner tube 124 has a first end connected to the first inner coupling ring 126a and a second end connected to a second inner coupling ring 126b. The second inner coupling ring 126b serves as a stationary locking element.

The stationary locking element 126b can include one or more locking protrusions 138 (four in the illustrated embodiment), which are oriented away from the inner tube 124 (as best shown in FIG. 7). The locking protrusions 138 can engage mating features on a housing portion 140 or 142 of the crimper mechanism 134, as further described below. The inner shaft assembly 120 can have a central longitudinal axis aligned with the longitudinal axis 136 of the assembly 104. The stationary shaft 122 can be made of any of various suitable materials, including metallic materials, for example, stainless steel. The inner tube 124 can be a cylindrical tube and forms an enclosed space extending from the first inner coupling ring 126a to the second inner coupling ring 126b. The inner tube 124 can be made of a transparent material, for example, a polycarbonate material.

FIG. 2 shows that the system 100 can include a back plate 186, which can include a central opening 188 for receiving an end portion 123 of the stationary shaft 122, and two peripheral openings 190 for receiving fasteners. As best shown in FIG. 14, the openings 190 can be aligned with openings 192 in the vertical support plate 172 and openings 194 in the actuator 102. Thus, fasteners 198 (FIG. 2) can be provided which extend through the openings 190, 192, and 194, thereby coupling the back plate 186, vertical support plate 172, and actuator 102 to one another. Further, the central opening 188 in the back plate 186 can comprise a non-circular shape substantially matching the shape of an enlarged head 196 of the stationary shaft 122. Thus, in this manner, the opening 188 can receive the enlarged head 196 and help to prevent it from rotating.

The outer crimper housing 106, best illustrated in FIGS. 8 and 9, can be coupled to the torque transmittal assembly 104 such that the actuator 102 can cause the housing 106 to rotate along its central longitudinal axis, which is aligned with the longitudinal axis 136 of the assembly 104. The housing 106 can include two circular end caps 128 and a cylindrical body 130. Each of the two end caps 128 can include four spiral cut grooves 132, and each of the grooves can be partially formed in an inner surface of the end cap 128. Each groove 132 can begin at a starting point near the periphery of the circular end cap 128 and extend generally in a spiral shape around the center of the end cap 128 until it terminates at an end point closer to the center of the end cap 128. At the center of each end cap 128, an opening 166 can be provided. As shown in FIG. 8, each end cap 128 can be provided with four spiral grooves 132, but in alternative embodiments, each end cap 128 can be provided with fewer or greater than four spiral grooves 132. Also in alternative embodiments, the grooves can be formed so as to extend completely through, rather than partially into, the end caps 128. Also in alternative embodiments, only one of the end caps 128 can be provided with spiral grooves. The end caps 128 can each be fastened to the cylindrical body 130 with a plurality of screws 168, thus making disassembly of the ends caps 128 from the body 130 relatively simple. Other embodiments use another type of quick- or easy-release fastener in place of or in addition to the screws 168, for example, latches, clasps, clamps, locking pins, cotter pins, captive screws, winged fasteners, knobs, handles, quick releases, and the like, which permits assembly and disassembly manually and/or using one or more hand tools.

FIG. 10 shows the stationary support 112, which can include a base 162, a vertical portion 164, and a plurality of locking protrusions 176 (four in the illustrated embodiment). The locking protrusions 176 are adapted to engage mating features on a housing portion 140 or 142 of the crimper mechanism 134.

FIGS. 11-12 show a disposable crimper mechanism 134, which is generally cylindrically shaped and which can be sized to fit within the outer crimper housing 106. The crimper mechanism 134 can include an outer housing comprising a first housing portion 140 and a second housing portion 142, and a plurality of crimping teeth 144. The first and second housing portions 140, 142 can be configured to fit together to form a substantially complete enclosure, which can house the plurality of teeth 144. Each of the first and second housing portions 140, 142 can include a plurality of radial slots 146, a plurality of radial ridges 150 on the inner surface of the housing portion, an opening 174 at its center, and a plurality of locking slots 160 formed in an annular wall surrounding the opening 174. Each of the crimping teeth 144 can include a pair of protrusions, or knobs 148, one disposed on either side of each of the crimping teeth 144, and a pair of grooves 152, one disposed on either side of each of the crimping teeth 144. As best shown in FIG. 11, the crimping teeth 144 can all have substantially the same configuration, and can have an overall wedge shape which tapers from a wide peripheral end to a relatively narrow central end.

The crimping teeth 144 can be enclosed within the first and second housing portions 140, 142 such that the knobs 148 extend into and interact with the radial slots 146 and extend beyond the surface of the crimper mechanism 134, with, the ridges 150 on the first and second housing portions 140, 142 also extending into and interacting with the grooves 152 in the crimping teeth 144. Thus, the crimping teeth 144 can be slidable within the crimper mechanism 134 along the axes defined by the slots 146, ridges 150, and grooves 152. In this configuration, a diameter of an aperture 158 at the center of the crimper mechanism 134 can be increased or decreased by controlling the movement of the crimping teeth within the enclosure formed by the first and second housing portions 140, 142. U.S. Pat. No. 7,530,253, which is hereby incorporated herein by reference, describes in further detail crimper mechanisms which can be used in the automated crimping system 100. In alternative embodiments, one of the first and second housing portions 140, 142 can independently be provided with slots and/or ridges in any combination, and the teeth 144 can each be provided with only one knob and/or groove, accordingly.

When the actuator 102, torque transmittal assembly 104, housing 106, stationary support 112, inner shaft assembly 120, and crimper mechanism 134 are assembled, the crimper mechanism 134 is housed within the outer housing 106 such that the knobs 148, which extend through the slots 146 and proud of, that is, laterally beyond the outer side surfaces of, the housing portions 140, 142, extend into and interact with the spiral grooves 132 of the end caps 128 of the housing 106. In this assembled configuration, the locking protrusions 176 of the stationary support 112 can extend through the opening 166 in an adjacent end cap 128 of the outer housing 106 and engage the locking slots 160 in the second housing portion 142 of the crimper mechanism 134. Similarly, the locking protrusions 138 of the stationary locking element 126b can extend through the opening 166 in an adjacent end cap 128 of the housing 106 and engage the locking slots 160 in the first housing portion 140 of the crimper mechanism 134. In this manner, the housing of the crimper mechanism 134 can be supported on one side by the stationary support 112 and on the other side by the stationary locking element 126b of the inner shaft assembly 120, thus ensuring that the housing portions 140, 142 of the crimper mechanism 134 remain rotationally stationary while the outer housing 106 is allowed to rotate therearound. Rotation of the outer housing 106 in turn causes radial movement of the crimping teeth 144, as further described below. The crimper mechanism 134 can be supported by the stationary support 112 and element 126b so that its central longitudinal axis is aligned with the longitudinal axis 136 of the assembly 104.

In use, a user can input a desired rotational sequence and/or crimping speed using the interface of the controller 108. The controller 108 controls movement of the actuator 102 in accordance with the commands inputted by the user. The actuator 102 causes the torque transmittal assembly 104 to rotate, thereby also causing the outer housing 106, and thus the spiral grooves 132 of the housing 106, to rotate about axis 136. The knobs 148 of the crimping teeth 144 interact with the spiral grooves 132 of end caps 128, but are constrained to move along the slots 146 of housing portions 140, 142. Thus, as the spiral grooves 132 rotate, the knobs 148, and thus the teeth 144, are forced to move radially in the direction of the slots 146. The direction of the rotation of the spiral grooves 132 thus can control the direction of the movement of the teeth 144 within the crimper mechanism 134 (e.g., radially inwards or radially outwards), and thus the diameter of the aperture 158.

Accordingly, the system described herein allows a user to control diameter and the rate of change of the diameter of the aperture 158. With appropriate programming of the controller 108, a user can cause the crimping teeth 144 and the aperture 158 to close, that is, to cause the diameter of the aperture 158 to decrease, or can cause the crimping teeth 144 and the aperture 158 to open, that is, to cause the diameter of the aperture 158 to increase.

In some embodiments, the length of the torque transmittal assembly 104, and/or the length of the inner shaft assembly 120 can be adjustable. In such embodiments, the length of these components can be adjusted by any of various suitable means in order to accommodate the insertion of delivery apparatuses of varying lengths into the system 100, as further described below. For example, the torque transmittal assembly and/or the inner shaft assembly can have a telescoping configuration comprising multiple nested cylindrical sections that can slide relative to each other to extend or retract the overall length of the assembly.

FIG. 13 illustrates various components of the system 100 in an assembled configuration, from a side view. FIG. 14 illustrates various components of the system 100 in an assembled configuration, from a cross-sectional side view. FIGS. 13 and 14 show how the illustrated components can be interconnected and coupled to one another in an assembled configuration.

FIG. 15 shows an alternative embodiment of an inner shaft assembly 200 and torque transmittal assembly 208 which can be used in the automated crimping system 100. The inner shaft assembly 200 can include a stationary shaft 202, which can be coupled to the actuator 102, and an inner tube 204, which can be coupled to an end portion of the stationary shaft 202. The stationary shaft 202 can be made of any of various suitable materials, including metallic materials, such as stainless steel, and the inner tube 204 can be made of any of various suitable transparent materials, such as a polycarbonate material or other transparent material, or non-transparent polymeric materials. The inner tube 204 can be provided with a plurality of stationary locking protrusions 206 that are adapted to engage locking slots 160 in one of the housing portions 140, 142.

The torque transmittal assembly 208 can include a pair of spaced apart flanges 210 which are coupled to one another by a cylindrical body or tube 212. The flanges 210 and tube 212 can be made of a variety of materials, including metallic materials, such as stainless steel. A plurality of windows 214 can be provided in the cylindrical tube 212 to provide visual access to the inside of the assembly 208. The inner shaft 200 can function in substantially the same manner as the inner shaft assembly 120. The inner shaft 200 can be coupled to and support one of the housing portions 140, 142 of the crimper mechanism 134, as described above, and can remain rotationally stationary while the torque transmittal assembly 208 is allowed to rotate around it. Similarly, the torque transmittal assembly 208 can be coupled at one end to the actuator 102 and at the other end to the outer housing 106, such that the actuator 102 can cause the outer housing 106 to rotate to effect movement of the crimping teeth 144.

### Methods of Using an Automated Crimping System

The automated crimping system 100 can be used to crimp prosthetic devices, for example, a prosthetic valve or a stent. The system 100 can provide various advantages over prior systems for crimping prosthetic valves. For example, the crimper mechanism 134 can be manufactured in a clean room, and packaged in a sterile package 300, shown in FIG. 16, for storage until the crimper mechanism 134 is used in the system 100. The crimper mechanism can be fabricated from a relatively inexpensive material, such as any of various polymeric materials, for example a molded and/or printed plastic, so that it can be disposed of and replaced on a regular basis. The crimper mechanism can also be fabricated from any of various suitable metallic materials (e.g., stainless steel), which can be cleaned, sterilized, and reused. The crimper mechanism can comprise a combination of materials, for example, polymeric and metallic.

Additionally, the crimper mechanism 134 can be configured to substantially contain and prevent liquid forced out of a prosthetic valve during crimping, for example, from the leaflets, from coming into contact with the surrounding components of the system 100, thereby reducing the need to clean the outer housing 106 and preventing contamination, corrosion, seizure, or any other damage to the system 100. Further, the outer housing 106 is easily disassembled by removing or releasing the screws 168 or other fasteners (for example, with a standard screwdriver or hex key), thus allowing for easy installation and replacement of the crimper mechanism 134, and easy cleaning of the housing 106 if and when needed. The housing 106 can further be made of a material selected to be compatible with any of various cleaning agents that may be used to clean the housing 106. As a result, the automated crimping system 100 can be reused many times, only requiring that the crimper mechanism 134 be replaced periodically, such as on a daily basis.

If desired, the system 100 can be situated within a laminar flow hood to protect users from any potentially harmful vapors. A crimper mechanism 134, packaged in a sterile packaging 300, can be removed from the packaging 300 and installed within the housing 106 of the system 100 such that the knobs 148 engage the spiral grooves 132, and such that the crimper mechanism 134 is supported by the stationary locking element 126b and the stationary support 112. If desired, a user can calibrate the system 100 by requesting, through the controller 108, that the crimper mechanism 134 be actuated such that the aperture 158 has a predetermined diameter. The user can then measure the diameter of the aperture 158 with a calibrated pin gauge 302, shown in FIG. 17, to ensure the actual diameter of the aperture 158 matches the requested and predetermined diameter. If the actual diameter matches the requested diameter to within appropriate tolerance levels, then the system 100 is ready to be used. If the actual diameter does not match the requested diameter to within appropriate tolerance levels, then the system 100 can be adjusted until it does.

Once the system 100 has been calibrated, the aperture 158 can be opened, and a prosthetic valve 306 can be positioned within the aperture 158. The aperture 158 can then be closed until the prosthetic valve 306 (FIG. 18) is partially crimped to a first partially crimped configuration. The aperture 158 can then be opened and the partially crimped prosthetic valve 306 can be removed from the crimper mechanism 134. The partially crimped prosthetic valve 306 can then be placed within a protective sleeve 304, and the partially crimped prosthetic valve 306, together with the protective sleeve 304, can be positioned within the aperture 158, as shown in FIG. 18. The aperture 158 can then be closed until sufficient pressure develops to ensure the prosthetic valve 306 and protective sleeve 304 can be held in place while a shaft 310 of a delivery apparatus (FIG. 19) is inserted through the prosthetic valve 306 and into the crimping apparatus.

FIGS. 19-20 show a shaft 310 which has been inserted through the prosthetic valve 306 and protective sleeve 304, and thus through the crimper mechanism 134 and outer crimper housing 106, and into the inner tube 124 (FIG. 20). The shaft 310 can be supported on the support pedestal 110, which can include a switch 308 for actuating a clamp within the support pedestal 110 for locking the shaft 310 in place. The switch 308 can be turned on, thereby actuating the clamp to hold the shaft 310 in place. As best illustrated in FIG. 20, the user can view a distal portion of the shaft 310 through the transparent inner tube 124 and the transparent outer tube 118, which can allow a user to view the shaft 310 while it is inside the crimper mechanism 134 and can help the user to ensure proper alignment of the various components.

The aperture 158 can then be closed until the prosthetic valve 306 is further crimped to a second partially crimped configuration. The aperture 158 can then be opened so that the prosthetic valve 306, protective sleeve 304, and shaft 310 can be removed from the crimper mechanism 134, as shown in FIG. 21. The protective sleeve 304 can then be removed from the prosthetic valve 306, and the prosthetic valve 306 and shaft 310 can be positioned once again in the aperture 158 of the crimper mechanism 134. The aperture 158 can then be closed until the prosthetic valve 306 is crimped to a final crimped configuration on the shaft 310. The aperture 158 can then be opened, the switch 308 turned off, thereby releasing the shaft 310 from the clamp of the pedestal 110, and the prosthetic valve 306 and shaft 310 can then be removed from the system 100, as shown in FIG. 22. The crimped prosthetic valve 306 and the distal end portion of the shaft 310 can then be placed within a protective sheath 314 and enclosed within a sterile package 312 for storage, as shown in FIG. 23. Thus, the prosthetic valve 306 can be stored and transported in a crimped configuration on a delivery apparatus in the package 312, until needed by a physician for implanting in a patient, thus eliminating the need for the physician to crimp the prosthetic valve 306 immediately prior to implantation.

In the illustrated example, the prosthetic valve 306 is crimped onto the shaft 310 of the delivery apparatus at a position proximal to an inflatable balloon 316 of the delivery apparatus. After being inserted into the vasculature of a patient, the prosthetic valve 306 can be moved onto the balloon 316 for deployment. In alternative embodiments, the prosthetic valve 306 can be crimped directly onto the balloon 316, or onto the shaft 310 distal of the balloon 316.

As noted above, the automated crimping system 100 can be configured for repeated use. Accordingly, the crimper mechanism 134 can be reuseable (e.g., can be sterilizable), disposable (e.g., replaceable), or both. The crimper mechanism 134 can be sterilized relatively easily due to its smaller size and material composition. Sterilization (prior to packaging or after being used) can be performed using ethylene oxide (ETO), gamma irradiation, or electron beam processing. The disposable crimper mechanism 134 can be disposed of after a predetermined number of crimping cycles. For example, the disposable crimper mechanism can be replaced after a single crimping cycle, or after 10 crimping cycles, or within a predetermined number of hours from the first time it was used, or after being used by workers for repeated crimping of prosthetic valves onto delivery catheters in one or more 8-hour shifts.

In some embodiments, automated crimping systems such as the system 100 can be capable of controlling the internal pressure of a balloon of a prosthetic valve delivery system, such as the balloon 316. For example, various crimper controllers such as the controller 108 can be provided with a port equipped with a Luer connector, which can be coupled to a lumen in the delivery catheter that in turn is in fluid communication with the balloon 316. In such an embodiment, the controller 108 can apply a pressure that is either greater than or less than atmospheric pressure in the balloon 316. For example, the controller 108 can control a pressurized fluid source (e.g., saline or compressed air) and/or a vacuum source to pressurize or to evacuate the balloon.

In some cases, the system 100 can be used to control the internal pressure of the balloon 316 during various steps of a prosthetic valve crimping process. For example, while the prosthetic valve 306 is being crimped onto the balloon 316, the controller 108 can be used to decrease the pressure in the balloon 316 to less than atmospheric pressure, thereby reducing the profile of the balloon 316 itself, which can also help to reduce the overall profile of the prosthetic valve 306 after it has been crimped.

As another example, once the prosthetic valve 306 has been crimped, the controller 108 can be used to increase the pressure in the balloon 316, thereby increasing its size. For example, FIG. 26 shows that after the prosthetic valve 306 has been crimped onto the balloon 316 on a shaft 310 of a delivery apparatus, the crimper controller 108 can be used to increase the pressure in the balloon 316, thereby partially inflating the balloon 316 and forming a distal bumper 318 distal to the prosthetic valve 306 and a proximal bumper 320 proximal to the prosthetic valve 306. The balloon can then be heat set so that these bumpers 318, 320 retain their shape when the delivery apparatus is disconnected from the pressurized fluid source and packaged for storage.

In some cases, after the prosthetic valve 306 has been crimped and removed from the crimper mechanism 134, it will recoil to a certain degree. That is, its diameter will increase from its final crimped diameter due to internal pressure exerted by compressed internal elements. For example, if the prosthetic valve 306 is crimped to a diameter of about 4 mm, it can recoil in some cases to a recoiled diameter of between about 6 mm and about 7 mm. In some embodiments, the diameters of the bumpers 318, 320 can be approximately equal to the recoiled diameter of the prosthetic valve 306, and the length of the bumpers can be between about 5 and about 10 mm. Consequently, the bumpers 318, 320 prevent the prosthetic valve 306 from sliding or shifting on the balloon 316. FIG. 26 shows that the balloon 316 can taper from the distal end of the prosthetic valve 306 to the shaft 310, and/or from the proximal end of the prosthetic valve 306 to the shaft 310, thereby forming a relatively smooth outer surface which can be advanced through a patient's vasculature.

### Methods for Safely Crimping a Prosthetic Valve

FIG. 24 illustrates an embodiment of a process 10 for assembling, crimping, and implanting an expandable prosthetic valve, which can be implemented, for example, using embodiments of the automated crimping systems disclosed herein. As shown at process block 12, the prosthetic valve is assembled. Implantable expandable prosthetic valves typically include at least a metal frame that makes up the structural part of the prosthetic valve and that is radially collapsible and radially expandable, and a set of leaflets that make up the functional part of the prosthetic valve and that are typically disposed within and secured to the frame. Such a frame may be formed from various materials. For example, the frame can be made of a plastically-expandable material that permits crimping of the prosthetic valve to a smaller profile for delivery and expansion of the prosthetic valve using an expansion device such as the balloon of a balloon catheter. Suitable plastically expandable materials include stainless steel and cobalt-chromium alloys. As another, alternative example, the frame can be made of a self-expanding material such as Nitinol. A self-expanding prosthetic valve can be crimped to a smaller profile and held in the crimped state with a restraining device such as a sheath covering the prosthetic valve. When the prosthetic valve is positioned at or near the target site, the restraining device can be removed to allow the prosthetic valve to self-expand to its expanded, functional size. The leaflets can similarly be formed from various materials, such as various biological materials (e.g., pericardial tissues) or biocompatible synthetic materials.

As shown at process block 14, the prosthetic valve, once assembled, can be treated with any one of a combination of various chemical agents that can help to prevent rejection of the prosthetic valve by the recipient, sterilize the prosthetic valve, stabilize proteins in the prosthetic valve leaflet tissue, make the tissue more resistant to mechanical fatigue, reduce degradation of the tissue by proteolytic enzymes, and/or allow packaging or delivery of the prosthetic valve in a dry form. In alternative embodiments, the leaflets can be treated with chemical agents prior to being secured to the frame. Once treated with appropriate chemical agents, the prosthetic valve can be crimped at process block 16 to a small profile, suited for implantation in a recipient. The prosthetic valve can be crimped directly onto a delivery device (e.g., on the balloon of a balloon catheter or on a shaft of a balloon catheter adjacent the balloon). Once crimped, the prosthetic valve can be packaged in a sterile package along with the delivery catheter (block 18) and then delivered to a healthcare facility (block 20). The prosthetic valve and the delivery catheter can be stored until it is needed for a procedure, at which point the physician can remove the prosthetic valve and the delivery catheter from the package and then implant the prosthetic valve in a patient (block 22).

FIG. 25 illustrates an embodiment of a more specific process 50 for crimping an expandable prosthetic valve. As shown in FIG. 25 at process block 52, the process 50 begins by receiving an expandable prosthetic valve in an expanded state, for example, a fully expanded state. The crimping process can continue by inserting the expandable prosthetic valve into a crimping system, for the example crimping system 100, at process block 54, and then partially crimping the expandable prosthetic valve at a first rate to a partially crimped configuration at process block 56. In particular embodiments, an expandable prosthetic valve can be considered partially crimped and process block 54 can accordingly be considered complete when the leaflets of the prosthetic valve begin to fold in on and contact other prosthetic valve components, such as the frame or the other leaflets.

The crimping process can continue at process block 58 by fully crimping the expandable prosthetic valve at a second rate to a fully crimped configuration. The second rate desirably is less than (i. e., slower than) the first rate. As the prosthetic valve is crimped from the partially crimped configuration to the fully crimped configuration, the leaflets of the prosthetic valve experience increasing pressure exerted by the crimping system, thus eluting interstitial fluid from the leaflets. Applicants have discovered that this phenomenon can induce shear forces in the tissue leaflets to cause tissue fiber rupture, and these forces increase with increasing viscosity of the interstitial fluid, as well as with increasing crimping rate, generally in accordance with Stokes' law. Due to this effect, crimping the expandable prosthetic valve at process block 58 from a configuration in which the leaflets begin to fold and contact other prosthetic valve components to a fully crimped configuration at a relatively slow rate can be advantageous, with the precise rate depending on the properties of the leaflet material, and on the viscosity of the interstitial fluid.

The crimping process can continue by removing the fully crimped prosthetic valve from the crimping system at process block 60. At the completion of any of the process blocks 52, 54, 56, and/or 58, the process can be paused for any appropriate period of time. That is, a succeeding process block need not begin immediately upon termination of a preceding process block. Further, in alternative processes, additional process blocks can be used. For example, in one possible alternative embodiment, an expandable prosthetic valve can be partially crimped to a first partially crimped configuration at a first rate, then partially crimped to a second partially crimped configuration at a second rate, then fully crimped at a third rate. In another alternative embodiment, the rate at which an expandable prosthetic valve is crimped can be continuously varied and determined based on the pressure resulting in the leaflets from the crimping process. In another alternative embodiment, an expandable prosthetic valve can be continuously crimped at a constant rate which approximates or is slower than the crimping rates described herein for fully crimping a prosthetic valve. Similarly, the crimping process can be terminated at (and thus the fully crimped configuration can be defined by) the point in the crimping process at which a pre-determined pressure is developed in the leaflets.

The process 50 can be used with a wide variety of prosthetic valves, as well as with a wide variety of crimping systems. As one specific example, the crimping process 50 can be used to crimp an expandable prosthetic valve having “dry” leaflets treated with glycerol (and thus having an interstitial fluid having a relatively high viscosity) and a diameter in its expanded configuration of about 78 French. The prosthetic valve can be partially crimped at a first rate to about 30 French, which can be completed in about 10 seconds or less. In this configuration, folds in the leaflets begin to contact each other and the metal frame. The prosthetic valve can then be further crimped from a configuration having a diameter of about 30 French to a fully crimped configuration having a diameter of about 14 French, which can be completed in some cases over the course of several minutes, for example, between about one and about three minutes.

The process of crimping a prosthetic valve and controlling the speed at which a prosthetic valve is crimped can be controlled and completed by any of various crimping systems. For example, a prosthetic valve can be crimped manually using a manual crimper (such as disclosed in U.S. Pat. No. 7,530,253), or automatically using an automated crimping system, for example any of the automated crimping systems described herein. A prosthetic valve can also be partially crimped using any of the crimping systems described herein in a first crimping step, and then in a second crimping step, removed from the crimping system and pulled through a crimping cone into a delivery sheath or a cylinder, which has an inside diameter equal to the final crimped diameter of the prosthetic valve, as described in U.S. Patent Publication No. 2012/0239142 to Liu et al., which is incorporated herein by reference.

Appropriate crimping systems can be driven by an electric motor or a combustion engine, can be pressure regulated, or can be pneumatically or hydraulically driven. Such a system can include various devices for collecting user input, such as buttons, levers, pedals, etc. In particular embodiments, the crimping system 100 can be programmed to crimp a prosthetic valve at one or more predetermined rates based on the expanded diameter and the desired final crimped diameter of the prosthetic valve. Thus, a prosthetic valve can be automatically crimped by activating the crimping system 100, which can carry out a predetermined crimping procedure.

The systems and methods disclosed herein can be used to avoid or minimize damage to the leaflets of an expandable prosthetic valve during the crimping process. The systems and methods disclosed allow such a prosthetic valve to be crimped in a controlled manner and thus allow the interstitial fluid to flow through the tissue of the leaflets in a controlled manner, thereby reducing shear stresses and maintaining the mechanical integrity of the leaflets. These systems and processes can improve the reliability, longevity, and performance of prosthetic valves, and can enable the crimping of such prosthetic valves to increasingly smaller profiles while allowing the treatment of prosthetic valves with various high-viscosity chemical compounds.

The systems and methods disclosed herein also allow automated crimping of a prosthetic heart valve, reducing ergonomic and repeatability issues in the crimping process. These systems and methods reduce the chance of operator fatigue and error, and improve the repeatability of crimping forces, rates, times, and specific crimping sequences, which can each be controlled by a computer. The systems and methods disclosed herein also allow the rapid sterilization and/or replacement of the components of the crimping system which contact the prosthetic valves being crimped and reduce the chance of accumulation of residue and the chance of corrosion and contamination of the system. The systems disclosed herein also allow for the simple disassembly of the system, thereby making cleaning, repair, and replacement of component parts more efficient.

For purposes of this description, certain aspects, advantages, and novel features of the embodiments of this disclosure are described herein. The disclosed methods, apparatuses, and systems should not be construed as limiting in any way. Instead, the present disclosure is directed toward all novel and nonobvious features and aspects of the various disclosed embodiments, alone and in various combinations and sub-combinations with one another. The methods, apparatuses, and systems are not limited to any specific aspect or feature or combination thereof, nor do the disclosed embodiments require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A”, “B”, “C”, “A and B” “A and C”, “B and C”, or “A, B, and C.”

As used herein, the term “coupled” generally means physically coupled or linked and does not exclude the presence of intermediate elements between the coupled items absent specific contrary language.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. An automated crimping system for crimping a prosthetic valve comprising:
a crimper controller;
a rotary actuator in communication with the controller;
an outer crimper housing coupled to the actuator and configured to rotate upon actuation of the actuator; and
a non-metallic crimper mechanism mounted within the outer crimper housing, the crimper mechanism comprising an inner housing and crimping teeth in the inner housing, the crimping teeth being operably coupled to the outer crimper housing such that rotation of the outer crimper housing effects movement of the crimping teeth.

2. The system of claim 1, further comprising a stationary support configured to support the inner housing in a stationary position.

3. The system of claim 2, wherein the stationary support comprises a plurality of locking notches which engage mating features of the inner housing.

4. The system of claim 2, further comprising a stationary inner shaft assembly extending from the rotary actuator to the inner housing and configured to support the inner housing in a stationary position.

5. The system of claim 4, wherein the stationary inner shaft assembly comprises a plurality of locking notches which engage mating features of the inner housing.

6. The system of claim 4, wherein the stationary inner shaft assembly comprises a transparent material.

7. The system of claim 1, further comprising a torque transmittal assembly coupled to the actuator and to the outer crimper housing, wherein the torque transmittal assembly transmits torque between the actuator and the outer crimper housing.

8. The system of claim 7, wherein the torque transmittal assembly comprises a transparent material.

9. The system of claim 1, wherein:
the outer crimper housing comprises a plurality of grooves having a spiral configuration; and
each of the crimping teeth comprises at least one knob constrained to move radially with respect to the crimper mechanism and extending into and interacting with the at least one groove.

10. The automated crimping system of claim 1, wherein the crimper mechanism is mounted within the outer crimper housing such that the crimper mechanism can be removed using only a screwdriver or hex key.

11. An automated method of crimping a prosthetic valve comprising:
inserting a prosthetic valve into an automated crimping system;
activating the automated crimping system to crimp the prosthetic valve; and
removing the crimped prosthetic valve from the automated crimping system.

12. The method of claim 11, wherein the act of activating the automated crimping system comprises:
partially crimping the prosthetic valve to a first partially crimped configuration;
placing the partially crimped prosthetic valve in a protective sleeve;
inserting a shaft of a delivery apparatus through the prosthetic valve; and
further crimping the prosthetic valve to a second crimped configuration.

13. The method of claim 12, wherein the shaft of the delivery apparatus is supported on a support pedestal.

14. The method of claim 12, wherein the act of activating the automated crimping system further comprises:
removing the protective sleeve from the prosthetic valve; and
further crimping the prosthetic valve to a third, final crimped configuration.

15. The method of claim 11, further comprising:
placing the prosthetic valve within a protective sheath; and
enclosing the prosthetic valve and protective sheath within a sterile package.

16. An assembly comprising:
a sterile crimper mechanism comprising a housing and a plurality of crimping teeth within the housing; and
a package containing the crimper mechanism;
wherein the crimper mechanism is adapted to be installed, once removed from the package, in an apparatus that can effect movement of the crimping teeth.

17. A method of crimping a prosthetic valve, comprising:
partially crimping the prosthetic valve at a first speed to a partially crimped configuration; and
further crimping the prosthetic valve at a second speed, wherein the second speed is slower than the first speed.

18. The method of claim 17, wherein the prosthetic valve is crimped at the second speed to a fully crimped configuration onto a delivery apparatus.

19. The method of claim 18, wherein partially crimping the prosthetic valve takes less than ten seconds and fully crimping the prosthetic valve takes more than sixty seconds.

20. The method of claim 19, wherein fully crimping the prosthetic valve takes more than two minutes.
